(19) United States
(12) Patent Application Publication
(10) Pub. No.: US 2022/0313086 A1
(43) Pub. Date: Oct. 6, 2022

(54) OPHTHALMOSCOPE FOR EXAMINING EYES

(71) Applicant: OCCYO GMBH, Innsbruck (AT)

(72) Inventor: Ulrich HAUSMANN, Innsbruck (AT)

(21) Appl. No.: 17/843,020

(22) Filed: Jun. 17, 2022

Related U.S. Application Data
(63) Continuation of application No. PCT/AT2020/060415, filed on Nov. 23, 2020.

(30) Foreign Application Priority Data
Dec. 19, 2019 (AT) A 60282/2019

(51) Int. Cl. A61B 3/14; A61B 3/00; A61B 3/113; A61B 3/125; A61B 3/12; A61B 3/107

(52) U.S. Cl. CPC A61B 3/14; A61B 3/0083; A61B 3/113; A61B 3/125; A61B 3/1216; A61B 3/107; A61B 3/1241; A61B 3/0008

(57) ABSTRACT

An ophthalmoscope for examining eyes, includes: a housing; a converting device for converting light into an electrical signal; and an objective, the objective comprising a lens and/or a mirror. The objective has a convexly curved focus area, a convexly curved image surface of an eye being able to be sharply imaged onto the converting device by means of the objective.

### BACKGROUND OF THE INVENTION

The invention relates to an ophthalmoscope for examining eyes, comprising a housing, at least one device for conversion for converting light into an electrical signal, and at least one objective, the at least one objective comprising at least one lens and/or at least one mirror. The invention further relates to such an ophthalmoscope with a slit lamp and to a use of such an ophthalmoscope on the human eye. Furthermore, the invention relates to a method for examining eyes and to a computer program product.

Commercially available ophthalmoscopes, which examine eyes mostly by using a slit lamp, can focus only on one plane. Since a complex eye geometry deviates from a plane, it is only possible to sharply image an annular zone of an eye onto the device for conversion, in particular at a magnification of the eye by a factor ten, where the remaining surface of the eye is imaged in a blurred manner by commercially available ophthalmoscopes. These ophthalmoscopes are unsuitable for an efficient and effective examination of eyes, and since a complete sharp image of the eye requires a plurality of images, each sharply imaging only one area of the eye, the eye would have to be positioned exactly the same over a period of all images, which corresponds to an ideal which is practically impossible to realize. Consequently, a reproducibility, a proper analysis, and a comparison through standardized images are simply not possible. To capture the required annular zone of the eye and the same annular zone of the eye over several images, if necessary for different eyes, is more difficult with commercially available ophthalmoscopes.

A system for inspection of an ophthalmic lens is already known from U.S. Pat. No. 6,577,387 B2, in which changes in an intensity of light of an image on a device for conversion of light—caused by changes in a thickness of the ophthalmic lens—are analyzed. The changes in the thickness of the ophthalmic lens are caused by cosmetic defects, irregularities, or by a design of the ophthalmic lens. An imaging lens arrangement in the form of an objective can have a curved focal surface in order to generate an improvement in focus regarding the curvature of the ophthalmic lens.

It is a disadvantage of the prior art that the inspection of ophthalmic lenses is performed by using an absorption technique, taking into account the Lambert-Beer's with ophthalmic lenses, and cannot be used for the inspection of eyes with a complex convex geometry, which is different from an ophthalmic lens. In addition, an absorption technique uses light with wavelengths in the ultraviolet range, in the infrared range, or laser light with monochromatic light, which are unsuitable for the examination of eyes for health reasons, at the power levels required for absorption technique. For visible light, additional optical elements would be required to manipulate light in order to be able to examine the inspection of the ophthalmic lenses using the absorption technique. Furthermore, it is not clear how images of ophthalmic lenses could be standardized for comparison based on specific parameters, so that a plurality of images could be used for analysis. A further disadvantage is that the light must first pass the ophthalmic lens in order to be able to draw conclusions about the characteristics of the ophthalmic lens on the basis of an absorption spectrum.

### SUMMARY OF THE INVENTION

Thus, the objectively technical object of the present invention is to provide an ophthalmoscope which is improved with respect to the prior art, in which the disadvantages of the prior art are at least partially eliminated, and which is characterized in particular by a reduced aberration, and allows reproducible as well as comparable images of the eye.

It is thus provided according to the invention that the at least one objective has a convexly curved focus surface, in which a convexly curved image surface of an eye can be sharply imaged by the at least one objective onto the at least one device for conversion, the entire visible area of the eye can be sharply imaged on the at least one device for conversion and a lens arrangement which has a negative Petzval sum is provided to image the convexly curved image surface of the eye.

The technical term focus surface of an objective refers to the surface that is generated when an object plane is projected through the objective. The focus surface can be used to display an image surface of an object surface on a device for conversion in a modified manner. If the object surface is convexly curved, then the image surface is also convexly curved without a lens arrangement.

A convexly curved focus surface of the lens arrangement changes the convexly curved image surface. A more convexly curved image surface can thus be transformed into a less convexly curved image surface.

The technical term object surface describes the surface geometry of an object, and, contrary to a flat object plane in this generalization, a curvature can be present in the surface geometry of the object. The technical term image surface describes the surface geometry of the image of the object, and, contrary to a flat image plane in this generalization, a curvature can be present in the surface geometry of the image of the object. This consideration is not limited to objectives with optical elements.

The term sharp in this context of ophthalmoscopy is defined to mean that the image of an object is of high resolution, and a contrast of a pair of lines between separate adjacent pixels on the at least one device for conversion is at least 20%, more advantageously at least 30%. In geometrical optics, only those points of the object can be reproduced as sharp image points in the device for conversion, which lie on an image plane, and are located at an object width to the lens. If the image plane is a convexly curved image surface, since the object surface of the object is also convexly curved, an arrangement of lenses must compensate for the curvature of the image surface in order to be able to generate a sharp image of the object on the device for conversion.

The convexly curved focus surface of the at least one objective lens enables complex convex geometries of an eye in the visible wavelength range of light to be sharply projected onto the at least one device for conversion, wherein the sharp image on the at least one device for conversion can be adapted to the given surface geometry of the at least one device for conversion by the convexly curved focus surface.

In addition, there is the positive characteristic that the convexly curved focus surface of the at least one objective can be individually adapted to the particularities of a specific eye geometry, and the entire surface of the eye visible to the at least one objective can be sharply imaged onto the at least one device for conversion.

Since the entire visible area of the eye can be sharply imaged on the at least one device for conversion, a standardization of images of the eye as well as a comparison of a plurality of images of the eye is also made possible, and a categorization based on eye-specific characteristics can be accomplished. An analysis of the image of the eye is thus considerably facilitated. Bidirectional measurements on the image of the eye are also made possible, which would not be feasible by imaging an annular zone of the eye.

If the ophthalmoscope with at least one lens comprises at least one mirror, it is a so-called catadioptric system, in which the sharp imaging of the eye is particularly favored by the at least one mirror.

The at least one mirror can be a freeform mirror, for instance.

Ophthalmoscopes are also possible, which at least one objective is formed by mirrors only, and it does not require lenses.

The at least one mirror can manipulate ray paths from the eye to the at least one device for conversion in a particularly favored manner in order to improve a sharp image of the eye.

In the following, the explanations refer to the image of one eye. In general, however, also both eyes can be imaged synchronously and simultaneously, respectively, in stereo by the ophthalmoscope.

As described above, protection is also requested for the ophthalmoscope with a slit lamp. Protection is also requested for the uses of the ophthalmoscope on the human eye.

Additionally, protection is also requested for a method of examining eyes, comprising the steps of
rough adjustment of a patient in a holding device, in particular with an accuracy below 3 mm,
recognition of a pupil and/or a vertex of a cornea of the eye by means of an image processing program, and it is particularly provided that the image processing program comprises an autofocus algorithm,
capturing at least one image of the eye by such an ophthalmoscope, where the at least one image of the eye is captured on at least one device for conversion.

The autofocus algorithm can perform a contrast optimization and/or position measurement, where the position measurement can be carried out, for instance, by a lateral camera to record a vertex of the eye as a line or a distance measurement, for instance, by a laser.

Furthermore, protection is also requested for a computer program product comprising commands, which, when executed by a computing unit, cause the computing unit to classify, for such an ophthalmoscope for examining eyes, at least one image of an eye from a memory unit, which is or can be brought into a data connection with the computing unit, wherein a classification is generated, based on a blood vessel structure and/or a corneal structure and/or a scar structure and/or an eye socket structure.

For instance, a blood vessel structure can be used as an indicator of redness of the eye and/or dryness of the eye.

According to an advantageous embodiment of the invention, the at least one device for conversion is in the form of a chip, preferably a planar chip, and it is preferably provided that the chip is in the form of a CMOS sensor.

The chip can be a CCD sensor, for instance. Particularly preferably, the chip is in the form of a CMOS sensor. The CMOS sensor comprises complementary metal-oxide-semiconductors, and can be present as an active pixel sensor or as a passive pixel sensor.

By means of a chip, and in particular by means of a CMOS sensor, an image of the eye can be conveniently read out within a very short time and made available for storage, thus increasing per time interval the possible number of images of the eye to be recorded by the at least one device for conversion.

In combination with a pulsed illumination and/or high transmission of the at least one objective, the time per image of the eye taken by the ophthalmoscope can be additionally reduced. Moreover, a high brightness can be achieved without blinding the patient. Furthermore, image noise is reduced.

Advantageously, the at least one device for conversion comprises at least one Bayer filter.

The technical term Bayer filter describes a color filter, where the three basic colors red, green and blue, and optionally the color white, are arranged in the form of pixels in a checkerboard structure.

The at least one Bayer filter enables the at least one device for conversion to capture color information of the image of the eye in a particularly favored manner.

The at least one Bayer filter can generate a splitting into red, green and blue pixels. Preferably, broadband light having a half-width in the range of 70 nm to 130 nm is transmitted through the Bayer filter. Maxima of a quantum efficiency of the Bayer filter are in the range between 450-455 nm for blue pixels, 535-545 nm for green pixels, and 610-615 nm for red pixels.

It has proved advantageous that at least two lenses are designed in the form of at least one air lens, where a Petzval sum of the at least one air lens is negative.

The term air lens describes a lens arrangement which has a negative Petzval sum. The Petzval sum equates to a reciprocal radius of a Petzval surface, where the reciprocal radius of the Petzval surface is defined by the sum of all reciprocal values of the involved refractive indices of the (thin) lenses multiplied by the respective focal length of the lens in the direction of radiation.

At least one air lens enables an image of a convexly curved surface on a planar surface with an objective, comprising a smaller number of lenses.

Furthermore, the imaging of the eye with a complex geometry, which for instance can be caused by variable radii of curvature, is particularly favored.

The exact numerical value of the required Petzal sum for an image of a convexly curved surface onto a planar surface can be calculated for instance via the further lenses in the objective, or distances between the at least one device for conversion and the object to be imaged, wherein the refractive indices and the focal lengths of the objective can be adapted to the requirements of the ophthalmoscope in this respect.

Preferably, a gas mixture with a refractive index smaller than 1.3, preferably smaller than 1.1, is arranged within the at least one air lens. And it is preferably provided that the gas mixture contains at least one noble gas, particularly preferably argon, nitrogen and/or air, particularly preferably in the form of the noble gas, nitrogen or air.

In general, lens arrangements are also possible which have a Petzval sum with the required value via gel-, liquid- and/or rubber-based inclusions. However, a gas mixture is particularly preferred to be completely enclosed by the at least two lenses. Air has proven to be particularly effective and cost-saving due to its low refractive index and high availability, and a particularly compact ophthalmoscope can be formed. However, other gas mixtures in the at least one air lens are also conceivable.

According to an advantageous embodiment of the invention, a lens-shaped fluid confinement is arranged between a sensor-sided lens and an object-sided lens, where the Petzval sum of the sensor-sided lens, the object-sided lens and the fluid confinement is negative.

Since the refractive index of the at least one fluid confinement can be easily and cost-effectively adjusted by the composition of a fluid, a high degree of flexibility is enabled with respect to the requirements for the at least one objective.

Furthermore, the refractive index of a fluid, such as air, is usually much lower than the refractive index of materials for lenses, such as optical glass, which means that the Petzval sum of a lens arrangement can be set to a negative value particularly efficiently, and/or with a low material input.

It has proved advantageous that the at least one air lens is designed in such a way that a lens with a first refractive index is arranged on the object side, and a lens with a second refractive index is arranged on the sensor side, and the second refractive index is higher than the first refractive index, and/or the at least one air lens is biconvex.

With a biconvex air lens, a combination of focal lengths is particularly favorable for a negative Petzval sum.

If the first refractive index is lower than the second refractive index, this can have a favorable effect on the ophthalmoscope in terms of negative terms in the Petzval sum.

An advantageous variant comprises having exactly two spaced-apart air lenses.

Two spaced air lenses allow additional optical elements, such as lenses in the objective, to be arranged between the two air lenses.

A ray path from the eye to the device for conversion can thus be manipulated by an arrangement and/or design of the air lenses and/or by additional optical elements, depending on the requirements for the image of the eye on the device for conversion.

It is particularly preferred that the at least one objective and/or an image on the object side and/or a ray path on the object side is essentially telecentric, preferably pericentric.

In conventional entocentric objectives, an angle of incidence becomes very shallow in a peripheral portion of a sclera, which results in a high decrease of a tangential resolution.

A more favorable tangential resolution is made possible by a telecentric objective, a telecentric object-sided image and/or a telecentric object-sided ray path. A tangential resolution of an even higher degree is made possible by a pericentric objective, a pericentric object-sided image and/or a pericentric object-sided ray path.

In one embodiment of the invention, an image on the at least one device for conversion is depicted in color.

By depicting the image in color, fine structures of the eye can be better identified by a user of the ophthalmoscope.

Preferably, the at least one device for conversion comprises an infrared Cut filter, and the infrared Cut filter particularly preferably cuts wavelengths above 620 nm.

According to a preferred embodiment of the invention, a sensor-sided numerical aperture of the at least one objective lies in the range from 0.04 to 0.1, preferably in the range from 0.06 to 0.08.

The numerical aperture indicates a capability of an optical element for the focusing of light. The numerical aperture is the product of the sinus of a half object-sided aperture angle and the refractive index of a material between the objective and a focus.

The numerical aperture on the sensor side in the range of 0.04 to 0.1 enables a resolution of the ophthalmoscope, which is not impaired by diffraction phenomena.

It has proved advantageous that the at least one objective has an aperture, and the aperture has a radius in the range from 2.5 mm to 3.5 mm, preferably in the range from 2.9 to 3.3.

A particularly preferred device for conversion comprises a resolution in the range of 4.88 μm. This is made possible by an aperture radius between 2.5 mm and 3.5 mm.

Furthermore, the ophthalmoscope can comprise exactly eight lenses and/or exactly two planar discs.

In order to project a complex geometry, such as the surface of the eye, onto a planar surface, an ophthalmoscope with eight lenses and two planar discs has proved particularly preferable in terms of the sharpness of the image of the eye, and taking the constructional dimensions as well as cost and weight into account.

The surface of the eye has convex sub-sections of different curvatures. The surface of the eye can be regarded as a freeform surface, which can be essentially rotation-symmetrical.

Alternatively, it is possible that at least one lens is aspherical.

Aspherical lenses make it possible to reduce aberration (imaging errors) particularly effectively due to the geometric structure of the aspherical lens.

According to an advantageous embodiment of the invention, at least one illuminated dot reticle is arranged in the at least one objective, preferably located centrally on an optical axis of the at least one objective.

By means of the at least one illuminated dot reticle, a fixation of the patient's eye can be achieved.

Preferably, the at least one illuminated dot reticle is arranged essentially on a focal surface of at least one lens, preferably at the focal point as the intersection of the focal surface with the optical axis.

This relaxes the eye and allows to accommodate to a point located at the infinite.

The at least one illuminated dot reticle provides a reference point for the eye, which can be targeted by the eye during a use of the ophthalmoscope. In the case of several images of the eye by the ophthalmoscope, a reference is thus provided in order to be able to image the same orientation of the eye over a period of time during which the images are taken. When multiple images are taken from different eyes, it is additionally possible to compare them with each other, since the at least one illuminated dot reticle ensures an essentially standardized alignment of the eye.

According to an advantageous embodiment of the invention, the at least one illuminated dot reticle comprises at least one diffractive structure, in which the at least one diffractive structure has an extension in the range of 25 μm to 75 μm, preferably in the range of 40 μm to 55 μm.

Particularly preferred, the at least one diffractive structure of the at least one illuminated dot reticle is illuminated laterally with at least one LED of any color, and the light of the at least one LED hits the eye to be imaged via the at least one diffractive structure.

According to an advantageous embodiment of the invention, the at least one illuminated dot reticle is covered by a mask on the sensor side.

By means of the mask, a disturbing false light, which is not used for imaging the eye by the at least one device for conversion, is reduced.

According to an advantageous embodiment of the invention, the housing comprises at least one peripheral cylinder, and the at least one peripheral cylinder is designed in a blackened manner, preferably in a portion of the at least one illuminated dot reticle.

The at least one blackened peripheral cylinder reduces scattered light that interferes with imaging the eye.

Advantageously, the light of the eye can be transmitted in chronological order through an objective lens, the illuminated dot reticle, a meniscus lens, an object-sided air lens, an object-sided diverging lens, the aperture, a converging lens, an achromat, a sensor-sided meniscus lens, a sensor-sided diverging lens, and a protective glass, and the eye light can subsequently hit the at least one device for conversion.

Thus, a ray path from the eye to the at least one device for conversion, in particular in the case of complex surface geometries of the eye and/or difficile structures, which may be present on the surface of the convexly curved object, deflected particularly effectively by the objective in such a way that a sharp image of the eye is generated on the at least one device for conversion.

It has proved favorable that the object lens and/or the object-sided meniscus lens and/or the achromat comprise flint glass.

Flint glass is characterized by an Abbe number lower than 50, and has a higher refractive index than crown glass. Flint glass also has a uniform optical dispersion.

According to an advantageous embodiment of the invention, the converging lens and/or the achromat comprise crown glass.

Crown glass is characterized by an Abbe number greater than 50 in color-corrected optical glasses, which keeps dispersion low, and a refractive index below that of flint glass.

It has proved advantageous that at least one holding device for a head is provided, preferably with a chin rest and/or a forehead support.

A holding device reduces a positional inaccuracy of the eye during at least one image by the ophthalmoscope and/or standardizes the at least one image of the eye. A chin rest and/or a forehead support can further increase the positional accuracy of the eye in order to also be able to use the images of the eye for a norming of images.

An advantageous variant is that the holding device is spaced from the at least one device for conversion in the range from 30 mm to 200 mm, preferably in the range from 40 mm to 100 mm.

When the at least one device for conversion can be brought into data connection with at least one computer, and the at least one computer can be brought into connection with a data base, the images captured by the device for conversion can be digitally saved for subsequent comparison or analyses.

It is particularly preferred that at least one illumination is arranged on the housing, and the illumination of the eye via the at least one illumination is in parts covered by the at least one objective.

By the at least one illumination itself, sufficient illumination of the eye is provided. Partial shielding of the at least one illumination by the at least one objective prevents that reflected light, directly on a smooth surface of the eye, does not pass through the at least one objective to the at least one device for conversion.

This enables an essentially homogeneous and/or isotropic illumination of the eye, wherein partial areas of overexposure are prevented without additional components of the ophthalmoscope.

Particularly preferably, the at least one illumination is in the form of an LED illumination.

The at least one illumination is preferably operated in a pulsed manner in order to avoid blinding a user of the ophthalmoscope and/or a patient. The pulse duration of the at least one illumination can be synchronized with the at least one device for conversion, and the at least one illumination illuminates in a time interval of an exposure time of the at least one device for conversion.

The exposure time of the at least one device for conversion can be set in a range from 1 μs to 5 s.

Preferably, the exposure time or/and pulse duration, respectively, is between 10 μs and 30 ms, particularly preferably between 20 μs and 200 μs. This particularly favors a brightness of the at least one image of the eye.

The at least one illumination is controlled with a pulse width modulation signal, where preferably a switch-on time of the at least one illumination is below 10%. This means that an illumination intensity can be increased tenfold while the patient's subjective perception of brightness remains the same.

Particularly preferred is at least one illumination that is pivotally positioned on the housing in order to ensure a fine adjustment of the illumination of the eye.

According to an advantageous embodiment of the invention, the at least one illumination is in the form of white light illumination, preferably with a sunlight-like white tone with a characteristic color temperature between 5000 K and 6000 K and/or a color rendering index of at least 95%, and/or fluorescent illumination.

With a fluorescent illumination, eyes or tear fluids on the eye, which for instance are enriched with a fluorescein dye, can be imaged in a particularly favorable manner, since the light of the fluorescent illumination can be emitted by the fluorescein dyes in a known wavelength range, and can be recorded by the at least one device for conversion.

It is particularly preferred that the fluorescent illumination has a wavelength range between 450 nm and 510 nm and/or between 750 nm and 780 nm.

This particularly favors a use of the ophthalmoscope on the human eye using the dyes fluorescein and/or indexyanine green for imaging the eye.

Thus, images of the eye are possible which particularly highlight specific portions of the eye. The fluorescent illumination is absorbed by the eye for instance at a wavelength of 765 nm, and a subsequent light emission, for instance by indocyanine green, takes place at 830 nm.

Enriching the eye with fluorescent dye can for instance be done by dropping fluorescent dye into the eye. An injection of the fluorescent dye for instance into the eye or blood vessels of the patient's body is also conceivable.

With white light illumination, visible light is used, and a fluorescent dye-enriched eye is not necessary for an image on the at least one device for conversion. The use of white light illumination is also less expensive in its use.

Particularly preferably, the ophthalmoscope is configured such that the ophthalmoscope has at least two modes, where one mode allows images of the eye to be captured via the white light illumination, and another mode allows images of the eye to be captured via fluorescent illumination. In this embodiment, the ophthalmoscope can switch between the modes.

It has proved advantageous that at least one fluorescence filter is arranged between the at least one device for conversion and the eye.

By means of the at least one fluorescence filter, which is particularly preferably arranged within the at least one objective in a focus of at least one lens, specific wavelength range's which are not necessary for imaging the eye, can be filtered out.

In one embodiment of the invention, the ophthalmoscope comprises at least one status screen, where at least one electronic information can be visualized on the at least one status screen.

Through the at least one status screen, a patient when using the ophthalmoscope can be informed, for instance, that the patient's eyelids are covering too much surface of the eye. The status screen can also display, for instance, a preview of the image by the at least one device for conversion. A display of electronic messages on the at least one status screen enables the patient, for instance, to position his/her eye in such a way that an image of the eye becomes as sharp as possible. Integrated color-coded LEDs are also conceivable for forwarding information to the patient and/or operator of the ophthalmoscope.

It has proved advantageous that the at least one image of the eye is read and/or saved on the at least one device for conversion on at least one computer, preferably in a database on the at least one computer.

By saving the at least one image on the computer, a duplication of the at least one image as well as a comparison of several images, possibly of different eyes, is made possible.

In general, a digital post-processing with a software is also possible in order to display the at least one image of the eye in a modified manner.

It is particularly preferable to provide that movements of the eye below 3 mm are tracked by an automated tracking, and/or an opening of an eyelid is detected by the image processing program. It is preferably provided that an opening below 12 mm, preferably below 16 mm, is displayed on at least one status screen.

This enables movements of the patient and/or overlaps by an eyelid not to impair the sharp image of the eye.

In a preferable variant of the invention, the at least one image of the eye is evaluated and/or analyzed regarding eye-specific characteristics.

This allows for a more efficient categorization of images of the eye and/or more effective classification based on specific eye characteristic parameters. In general, this can be supported by computerized data processing software.

Eye-specific characteristics can be, for instance, dimensions and/or geometries of the eye, a blood vessel structure, a corneal structure, a scar structure, and/or an eye socket structure.

An advantageous variant consists of categorizing the at least one image of the eye on the at least one computer for standardization and/or norming.

Categorization facilitates a search for eye-specific features and/or a comparison of similar eye-specific features in the at least one image of the eye.

In one embodiment of the invention, the eye has a contact lens and a tear fluid enriched with fluorescent dye. The tear fluid is spotlighted with a fluorescent illumination, the tear fluid emits light in a wavelength range between 515 nm and 530 nm and/or between 825 nm and 835 nm, and a distribution of the emitted light of the tear fluid between the contact lens and the eye is recorded on the at least one device for conversion.

The imaged distribution of the tear fluid through the image of the eye, in which the imaged distribution is caused by a light emission via the fluorescent dyes (for instance fluorescein and/or indexyanine green), makes it possible to identify fitting inaccuracies of the contact lens on the eye.

In one embodiment of the invention, the eye comprises at least one fluorescent dye and a blood vessel structure and/or a lymph vessel structure and/or a corneal epithelium of the eye, preferably via the at least one fluorescence filter, is imaged on the at least one device for conversion and/or saved on the at least one computer.

The blood vessel structure, the lymph vessel structure and/or the corneal epithelium can be imaged by means of visible light. These structures are particularly favorably imaged by the use of the fluorescent illumination, since the contrast to further structures of the eye is increased.

It has proved advantageous that the fluorescent dye comprises fluorescein and/or indexyanine green.

As a result, structures of the eye can be imaged in a particularly favorable manner by the at least one device for conversion.

It is particularly preferable that the method is carried out again after a period of at least one day. This makes it possible to make a state of the eye in the course of time visible.

In particular, this method is used in telemedicine. For instance, an image of an eye can be captured by the ophthalmoscope at a first location, and transmitted to a second location, which for instance can be a central ophthalmic practice.

At specific time intervals, further images of the eye can thus be captured and sent to the second location, where, when saved, a plurality of images of the eye are present, which make the state of the eye visible over a course of time.

For instance, via images of the eye in a database, neovascularizations can become visible over a period of time, and a localization and/or an extent of the vascular neoplasm can be detected.

An explicit appointment with an ophthalmologist is no longer necessary. The ophthalmologist can make an assessment based on the acquired data, and the ophthalmoscope provides an assistance function in terms of data preparation for an improved diagnosis by the ophthalmologist.

It has proved advantageous that an image overlay with automatic blood vessel detection and/or automatic limbus detection can be used to control the classification.

This particularly favors an analysis of the images of the eye.

It has proved advantageous that a semi-automatic lesion detection and/or bidirectional measurements can be carried out.

It is particularly preferable that standardized images of at least one iris are saved in the memory unit.

In one embodiment of the invention, a color of a contact lens and/or an artificial iris is selected, depending on a color of two iris, and the color of the contact lens and/or the artificial iris is adapted to one of the two iris.

In the case of different iris colors, a colored contact lens and/or a colored artificial iris can balance out the different color tones of the iris.

In one embodiment of the invention, the blood vessel structure is automatically recognized, preferably via artificial intelligence.

For instance, a blood vessel structure of the eye can be recognized or identified particularly effectively via machine learning. The blood vessel structure can subsequently be used for an analysis and/or a categorization.

It has proved advantageous that a quantification of changes in the blood vessel structure and/or the corneal structure is calculated via the image overlay.

With a plurality of images of the eye, it can be recognized via image overlay how much eye-specific characteristics, for instance blood vessel structure, have changed over a course of time.

The quantification can hereby indicate the temporal change in the form of a key figure or eye-specific parameters, such as eye redness.

It is particularly preferably provided that a red pixel density measurement is carried out. The red pixel measurement integrates a total red portion of the at least one image, and/or calculates an area portion of the blood vessel structure at the at least one image.

If the total red portion of the pixels of at least one image is integrated, a measure of eye redness can be stated quickly and simply.

If the area portion of the blood vessel structure is calculated, the measure of eye redness can be stated more precisely. For instance, the blood vessel structure can be determined using a threshold value of the red portion of the pixels in the image of the eye.

If the computer program product comprises an automatic lesion detection of the eye, anomalies of the eye can be automatically detected and displayed, for instance, by means of a visualization.

An advantageous variant is that an opening of the eye of at least 12 mm, preferably at least 16 mm, is detected and, if the opening is less than 12 mm, preferably less than 16 mm, an electronic information is transmitted to the at least one status screen.

This allows the eye not to be covered to a large extent by the eyelid when the eye is imaged. Via the electronic information, the patient is informed to open the eye wider when using the ophthalmoscope in order to be able to image more aspects of the ocular surface.

Smaller misalignments of the eye can be corrected by the ophthalmoscope, for instance by tracking, and in case of too high misalignments—of any kind—the status screen indicates an insufficient alignment and/or exposure of the eye.

If a sharp image of the eye is possible, the patient can be informed via the at least one status screen, preferably via an LED and/or a visualization and/or an electronic message, that the sharp image of the eye can be carried out when maintaining the position. A preview of the image of the eye via the at least one status screen is also possible.

The ophthalmoscope can automatically perform an image of the eye when the eye is properly positioned and not covered over a large area. In addition, a viewing direction apart from the at least one illuminated dot reticle can be detected and/or displayed on the at least one status screen.

### BRIEF DESCRIPTION OF THE DRAWINGS

Further details and advantages of the present invention are explained in more detail below by means of the figure description with reference to the embodiments shown in the figures. Therein, they show:

FIGS. 1a-1c show an ophthalmoscope according to a preferable embodiment with a holding device,

FIGS. 2a-2c show the ophthalmoscope according to the embodiment shown in FIGS. 1a-1c with a slit lamp,

FIGS. 3a-3d show ray paths of light between an eye and a device for conversion, and the ray paths pass through an objective,

FIG. 4 shows an objective with an illumination in the direction of an eye in a schematically indicated view from the side,

FIG. 5 shows the ophthalmoscope according to the embodiment shown in FIG. 1a in data connection with a computer and a flow chart,

FIG. 6a shows an image not according to the invention of a concave geometry through a lens with a depicted correlation between a focus surface, an image surface, an object-sided image, and an image,

FIG. 6b shows an objective of an ophthalmoscope with three mirrors during capturing an image of an eye onto a device for conversion,

FIGS. 7a-7b shows a blood vessel structure in an image of a human eye, and a blood vessel structure, identified by a computer program product, using the image of the eye.

### DETAILED DESCRIPTION OF THE INVENTION

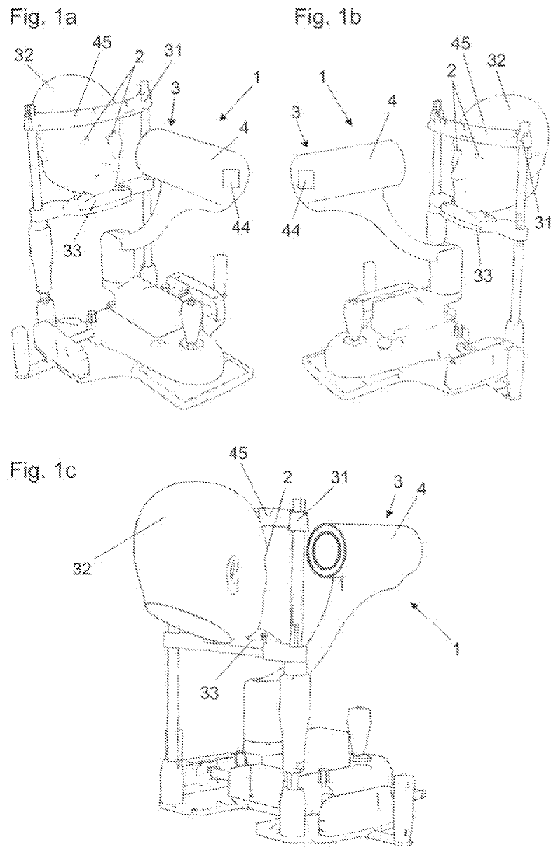

FIG. 1a shows an ophthalmoscope 1 for examining eyes 2 comprising a housing 3 and a device for conversion 4 of light into an electrical signal (not visible in the illustration).

A holding device 31 for a head 32 is provided, and a chin rest 33 and a forehead support 45 are arranged on the holding device 31. The head 32 is aligned to the ophthalmoscope 1 by the holding device 31 and the chin rest 33 as well as the forehead support 45 in such a way that the eye 2 is positioned stationary for at least one image 16 by the ophthalmoscope 1.

The holding device 31 is spaced from the at least one device for conversion 4 between 30 mm and 200 mm, and the spacing can be adjusted by an operator of the ophthalmoscope 1.

The ophthalmoscope 1 is used on the human eye 2.

FIG. 1b shows the ophthalmoscope 1 according to FIG. 1a pivoted about a vertical axis by 90 degrees in one direction. FIG. 1c shows the ophthalmoscope 1 according to FIG. 1a pivoted about a vertical axis by 90 degrees in the opposite direction.

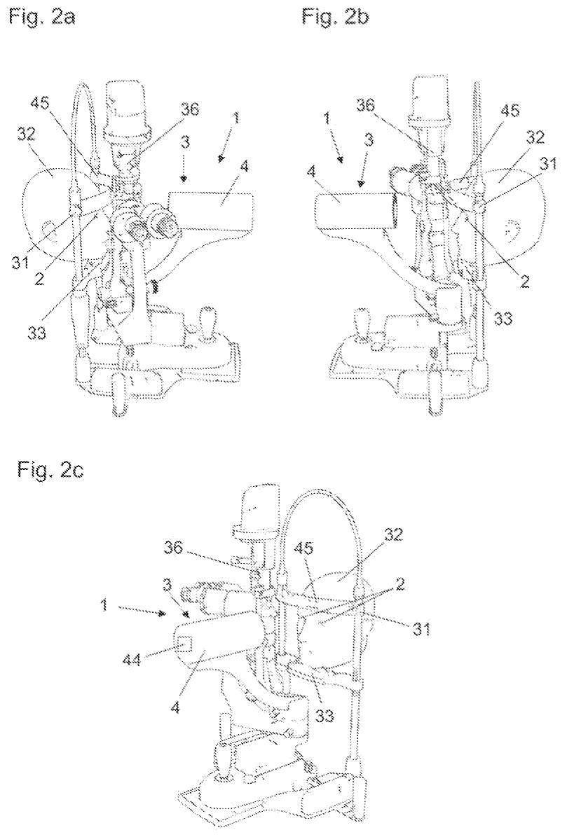

FIG. 2a shows the ophthalmoscope 1 according to FIG. 1a with a slit lamp 36.

An image 16 on the at least one device for conversion 4 can be displayed in color. A sensor-sided numerical aperture of the objective 5 lies in the range of 0.04 to 0.1. The objective 5 has an aperture, and the aperture has a radius in the range of 2.5 mm to 3.5 mm during imaging the eye 2 by the ophthalmoscope 1 (not shown for clarity reasons).

The objective can also have an object-sided numerical aperture, preferably in the range of 0.04 and 0.1.

FIG. 2b shows the ophthalmoscope 1 according to FIG. 2a pivoted 180 degrees about a vertical axis. FIG. 2c shows the ophthalmoscope 1 according to FIG. 2a in a perspective view from the front.

Where the eye 2 has a contact lens and a tear fluid enriched with fluorescein dye as a fluorescent dye, in which the tear fluid is illuminated with a fluorescent illumination 35, the tear fluid can emit light in a wavelength range between 515 nm and 530 nm, and a distribution of the emitted light of the tear fluid between the contact lens and the eye 2 is recorded on the at least one device for conversion 4. If the fluorescent dye is indocyanine green, the emitted light has a wavelength in the range of 825 nm to 835 nm.

If the eye 2 comprises at least one fluorescent dye, a blood vessel structure 46 and/or a lymph vessel structure and/or a corneal epithelium of the eye 2 can be imaged on the at least one device for conversion 4. Particularly preferable hereby is the use of a fluorescence filter 43 (not shown for reasons of clarity). The image 16 can subsequently be saved on a computer 34.

The ophthalmoscope 1 comprises a status screen 44, in which electronic information is visualized on the status screen 44.

The images 16 can be repeated after a desired interval.

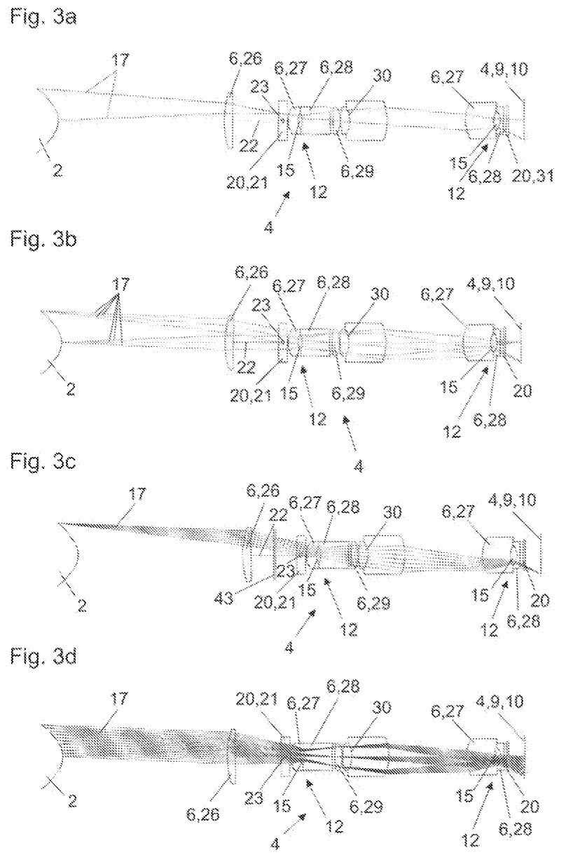

FIG. 3a shows a sectional view of a surface geometry of an eye 2, an objective 5, and a device for conversion 4. The device for conversion 4 is formed in the form of a planar chip 9, and the chip 9 is formed as a CMOS sensor 10. The CMOS sensor 10 comprises a Bayer filter 11 (not shown for clarity reasons).

The objective 5 comprises eight lenses 6 and two planar discs 20. The lenses 6 are formed spherical, but can generally also be formed aspherical.

A ray path 17 of light, starting from the surface geometry of the eye 2, impinges on the CMOS sensor 10 after a transmission through the objective 5. The light of the eye 2 is transmitted in chronological order through an objective lens 26, an illuminated dot reticle 21, a meniscus lens 27, an object-sided air lens 6, an object-sided diverging lens 28, an aperture, a converging lens 29, an achromat 30, a sensor-sided meniscus lens 27, a sensor-sided diverging lens 28, and a protective glass.

Two such ray paths 17 are shown in the illustration, in which—starting from the eye 2—the upper ray path 17 starts from a more curved portion of the surface geometry of the eye 2, and the lower ray path 17 starts from a less curved portion of the surface geometry of the eye 2. Due to the transmission through the eight lenses 6, the upper ray path impinges on a lower portion, and the lower ray path impinges on an upper portion relative to the lower portion of the CMOS sensor 10.

One planar disc 20 comprises the illuminated dot reticle 21, and the second planar disc 20 acts as a protective glass for the device for conversion 4.

Four lenses 6 are formed in the shape of two air lenses 12 spaced apart from each other, and a Petzval sum of the two air lenses 12 is negative. The two air lenses are formed in a biconvex manner.

The two air lenses 12 each comprise a lens-shaped fluid confinement 15, and the lens-shaped fluid confinement 15 is arranged between a sensor-sided lens 6 and an object-sided lens 6. The Petzval sum of the sensor-sided lens 6, the object-sided lens 6, and the fluid confinement 15 is negative.

The air lens 12 is designed in such a way that a lens 6 with a first refractive index is arranged on the object side, and a lens 6 with a second refractive index is arranged on the sensor side, and the second refractive index is higher than the first refractive index.

FIG. 3b shows the eye 2, the objective 5 as well as the device for conversion 4 according to the embodiment example shown in FIG. 3a, in which to the upper and lower ray path 17 in each case two further ray paths 17 are shown, which each have a smaller or larger aperture angle to a longitudinal alignment of the objective 5. Despite the different aperture angles, identical points on the surface geometry of the eye 2 meet identical points on the CMOS sensor 10 after transmission through the objective 5.

As ray paths 17 of equal points of the eye 2 of different aperture angles are projected onto equal points on the device for conversion 4, this results in a sharp image 16 of the eye 2 on the device for conversion 4, and the sharp image 16 is not limited to an annular zone of the eye 2.

The reason for the sharp image 16 of the eye 2 on the CMOS sensor 10 is a convexly curved focus surface 7 (not shown for clarity reasons) of the objective 5. A convexly curved image surface 8, which, without the objective 5, would correspond to the convexly curved surface geometry of the eye 2, is sharply imaged onto the device for conversion 4 by the objective 5 with the convexly curved focus surface 7. A sharp image 16 of the eye 2 on the CMOS sensor 10 is the result of this constructive design of the objective 5.

FIG. 3c shows the objective 5 according to FIG. 3a, in which different ray paths 17, starting from a point on the surface of the eye 2, which are each imaged by the objective 5 onto the same point on the CMOS sensor 10. A blurred image 16 is thereby prevented.

The object lens 26, the object-sided meniscus lens 27, and the achromat 30 comprise flint glass. The converging lens 29 and the achromat 30 comprise crown glass.

The objective 5 can also comprise one or more mirrors 42 (not visible in the illustration) to direct the ray paths 17 particularly favorably to the CMOS sensor 10 and to improve a sharp image 16 of the eye 2.

A fluorescence filter 43 is arranged between the at least one device for conversion 4 and the eye 2.

The fluorescence filter is particularly preferably placed in the focus of one of the lenses 6.

A pupil aperture can be located between the diverging lens 28 and the converging lens 29.

FIG. 3d shows the objective 5 according to FIG. 3a, in which it is visible that the objective 5 and an object-sided image 16a (not shown for clarity reasons) and an object-sided ray path 17 are essentially formed telecentrically. The objective 5, the object-sided image 16 and the object-sided ray path 17 can also be formed pericentrically.

In order to fix over an alignment of the eye 2 over a longer period of time relative to the objective 5, the illuminated dot reticle 21 is arranged in the at least one objective 5, centrally located on an optical axis 22 of the at least one objective 5. This also allows for a norming of the images 16 of the eye 2, as the alignment of the eye 2 can be directed again to the desired position after an interruption of images by the ophthalmoscope 1.

The illuminated dot reticle 21 comprises a diffractive structure 23, in which the diffractive structure 23 has an extension in the range of 25 μm to 75 μm. The illuminated dot reticle 21 is covered on the sensor side by a mask 24 (not shown for clarity reasons) so as not to interfere with the image 16 of the eye 2 on the device for conversion 4. The diffractive structure 23 can be illuminated laterally with an LED of any color, and the light from the LED is redirected towards the eye.

An optical length of the ray paths is equal. To achieve this, an optical density is increased at the periphery, and decreased at the center. This compensation can be generated by biconvex air lenses 12.

The objective 5 is constructively designed in such a way that an optical transmission is homogeneous and particularly high over the entire field.

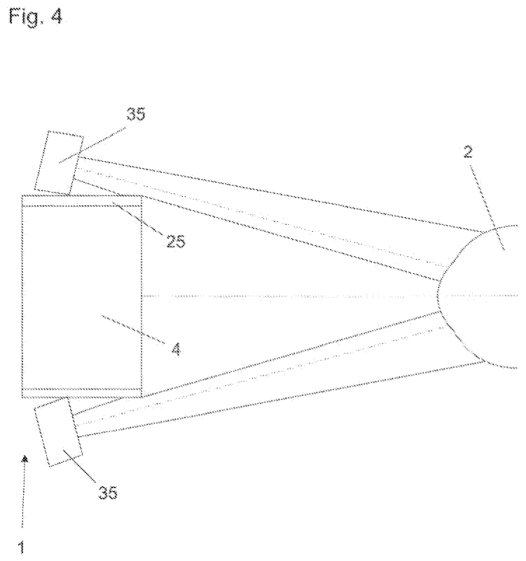

FIG. 4 shows an objective 5, in which the housing 3 comprises a peripheral cylinder 25. The peripheral cylinder 25 is formed in a blackened manner. It can also be provided that the peripheral cylinder 25 is formed in a blackened manner only in a portion of the illuminated dot reticle 21.

An illumination 35 is arranged on the housing 3, in which the illumination of the eye 2 via the illumination 35 is covered partially by the objective 5. In general, the shape of the illumination 35 is arbitrary. Particularly preferable is an illumination 35 which is arranged annularly around the housing 3. However, several illuminations 35 can also be arranged on the housing 3, or an illumination 35 in the form of a commercial lamp can be used.

That illumination of the eye 2 which is covered by the objective 5 would result in unwanted reflections on the device for conversion 4, which would additionally result in unwanted overexposure of portions of the eye 2. In addition, the covered portion of the illumination can reduce pupil dilation and/or pain sensation. Central portions of a cornea are thus not illuminated directly, but this portion of the illumination is deflected or shadowed.

The illumination 35 is designed such that the light of the illumination 35, which is diffusely scattered upon penetrating the cornea and a sclera, contributes to the image 16.

The light of the illumination 35 increases in the direction of an outer region of the eye 2 in order to generate a homogeneous brightness distribution on the device for conversion 4.

The illumination 35 can be present in the form of white light illumination, for instance with a sunlight-like white tone with a characteristic color temperature between 5000 K and 6000 K and/or a color rendering index of at least 95%. The illumination 35 can comprise polychromatic light. The illumination 35 can also be designed as a fluorescent illumination, for instance with a wavelength range between 450 nm and 510 nm and/or between 750 nm and 780 nm.

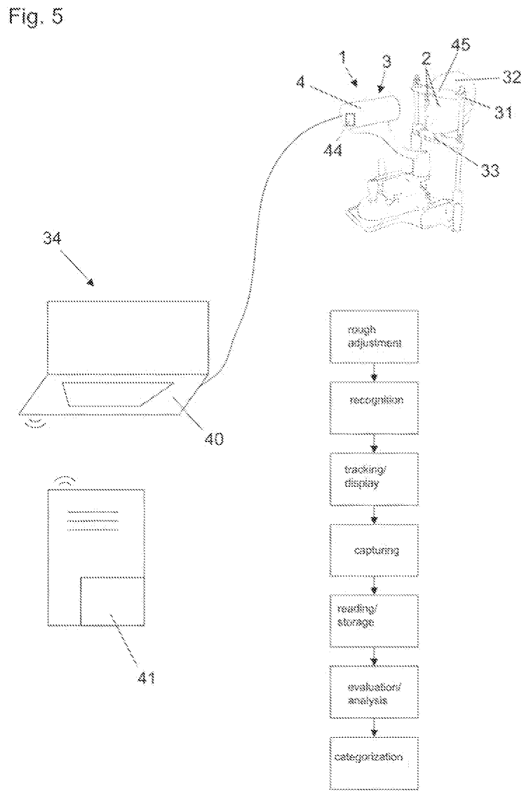

FIG. 5 shows an ophthalmoscope 1, wherein the device for conversion 4 of the ophthalmoscope 1 is in direct data communication with a computer 34. The computer 34 is in signal-transmitting data connection with a database on a remote computer. However, the database can also be present directly on the computer 34.

A flowchart describes a method for examining eyes 2, comprising the following steps:
rough adjustment of a patient in a holding device 31, in which an accuracy of the rough adjustment below 3 mm is particularly advantageous for sharp images of the eye 2 on the device for conversion 4,
recognition of a pupil and/or a vertex of a cornea of the eye 2 via an image processing program, and the image processing program can comprise an autofocus algorithm to support the recognition, and
capturing at least one image 16 of the eye 2 by the ophthalmoscope 1, in which the at least one image 16 of the eye 2 is captured on the device for conversion 4.

In addition, further steps are being provided according to this shown embodiment of the method:
movements of the eye 2 below 3 mm can be tracked by automated tracking and/or an opening of an eyelid can be detected via the image processing program—when the opening of the eye 2 is at least 12 mm, preferably at least 16 mm, an indication can be displayed on a status screen 44,
the at least one image 16 of the eye 2 on the device for conversion 4 is read into the computer 34 and saved,
the at least one image 16 of the eye 2 is evaluated and analyzed regarding eye-specific characteristics, and
the at least one image 16 of the eye 2 is categorized at the computer 34 for standardization and norming.

A computer program product is provided at the computer 34, which comprises commands that, when carried out by a computing unit 40, cause the computing unit 40 to do the following for an ophthalmoscope 1 for examining eyes 2:
to classify the at least one image 16 of the eye 2 from a storage unit 41, which is in a data connection with the computing unit 40, and a classification is generated based on a blood vessel structure 46 or a corneal structure or a scar structure or an eye socket structure,
in which an image overlay with automatic blood vessel detection or automatic limbus detection is used to control the classification
in which a semi-automatic lesion detection or bidirectional measurements are carried out, and
in which standardized images 16 of at least one iris are saved in the memory unit 41.

The computer program product can also select a color of a contact lens and an artificial iris depending on a color of two iris, and the color of the contact lens and the artificial iris is matched to one of the two iris.

In addition, the computer program product can automatically detect the blood vessel structure 46, and hereby using, for instance, artificial intelligence. A quantification of changes in the blood vessel structure 46 and/or the corneal structure can also be calculated via the image overlay.

In addition, it is possible to perform a red pixel density measurement, and the red pixel measurement integrates a total red portion of the at least one image 16, and/or calculates an area portion of the blood vessel structure 46 on the at least one image 16.

The computer program product comprises an automatic lesion detection of the eye 2, and can detect an opening of the eye 2 of at least 12 mm. With an opening of less than 12 mm, an electronic information is transmitted to the at least one status screen 44 of the ophthalmoscope 1.

The ophthalmoscope 1 can be used in a stand-alone embodiment or as an add-on embodiment for a commercial slit lamp 36.

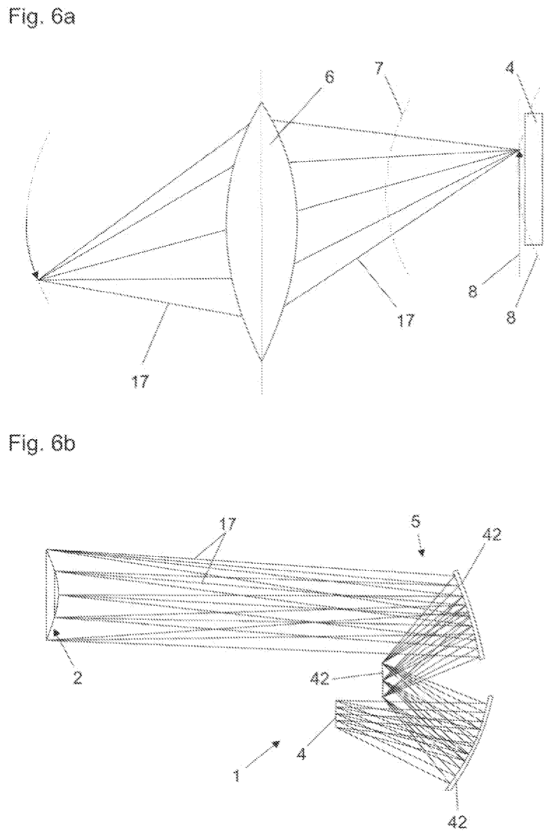

FIG. 6a shows a concavely curved object with a concavely curved object surface and a device for conversion 4. Ray paths 17 visualize the transmission of light through a lens 6, starting from the concavely curved object in the direction of the device for conversion 4.

Without lens 6, the concavely curved object surface would be imaged onto a concavely curved image surface 8 (indicated by a dashed line). The lens 6 enables an image 16 on a planar device for conversion 4, in which the image surface 8 is also designed to be planar (indicated by a dashed line). The concavely curved image surface 8 can thus be sharply imaged onto the device for conversion 4 through the lens 6. With a convexly curved object, a convexity would be further enhanced by this lens 6, making a sharp image 16 onto the planar device for conversion 4 impossible.

The focus surface 7 is defined to the right of the lens 6, and in the illustration, the focus surface 7 is indicated by a dashed line in the image plane. The focus surface 7 is designed to be concave, and a convex focus surface 7 is provided according to the invention.

FIG. 6b shows ray paths 17 between an eye 2 and a device for conversion 4. The ray paths 17 are redirected to the device for conversion by three mirrors 42.

The mirrors 42 are hereby designed as free-form mirrors. However, the mirrors 42 can also comprise other types of mirrors.

The objective 5 of the ophthalmoscope has a curved focus surface 7 (not shown for clarity reasons) in order that the eye 2 can be sharply imaged on the device for conversion 4.

Using the objective 5 on the eye 2 in combination with at least one lens 6 is also possible.

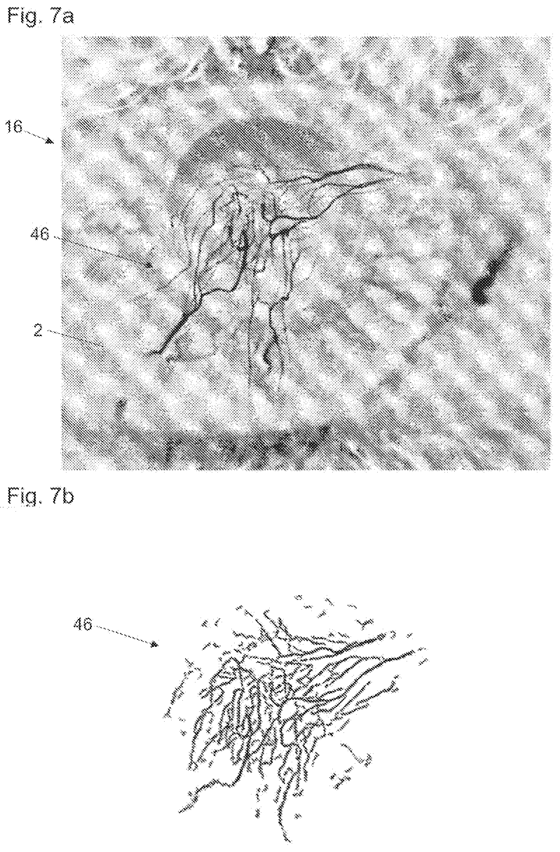

FIG. 7a shows an image 16 of a human eye 2. A blood vessel structure 46 can be detected, in which the blood vessel structure 46 can be identified by a reddish color.

FIG. 7b shows a blood vessel structure 46, which is generated over the image 16 of the eye 2 shown in FIG. 7a by the computer program product using the automatic blood vessel detection.

Based on the blood vessel structure 46, an analysis, a categorization and/or a red pixel density measurement of the image 16 of the eye can be performed.

1. An ophthalmoscope for examining eyes, comprising
a housing,
at least one device for conversion for converting light into an electrical signal, and
at least one objective, wherein the at least one objective comprises at least one lens and/or at least one mirror,
wherein
the at least one objective having a convexly curved focus surface, wherein a convexly curved image surface of an eye being able to be sharply imaged onto the at least one device for conversion by means of the at least one objective,
the entire visible area of the eye can be sharply imaged on the at least one device for conversion and
a lens arrangement which has a negative Petzval sum is provided to image the convexly curved image surface of the eye.

2. The ophthalmoscope according to claim 1, wherein the at least one device for conversion is provided in the form of a planar chip or the at least one device for conversion comprises at least one Bayer filter.

3. The ophthalmoscope according to claim 1, wherein at least two lenses are formed in the shape of at least one air lens, wherein a Petzval sum of the at least one air lens is negative or a gas mixture with a refractive index smaller than 1.3 is arranged within the at least one air lens, wherein the gas mixture contains at least one noble gas, nitrogen or air.

4. The ophthalmoscope according to claim 1, wherein a lens-shaped fluid confinement is arranged between a sensor-sided lens and an object-sided lens, wherein the Petzval sum of the sensor-sided lens, the object-sided lens and the fluid confinement is negative.

5. The ophthalmoscope according to claim 3, wherein the at least one air lens is designed such that a lens with a first refractive index is arranged on the object side, and a lens with a second refractive index is arranged on the sensor side, wherein the second refractive index is higher than the first refractive index, or the at least one air lens is designed in a biconvex manner or exactly two spaced-apart air lenses are present.

6. The ophthalmoscope according to claim 1, wherein
the at least one objective or an object-sided image or an object-sided ray path is designed to be essentially telecentric or pericentric, or
an image on the at least one device for conversion is shown in color, or
a sensor-sided numerical aperture of the at least one objective lies in the range from 0.04 to 0.1, or
the at least one objective has an aperture, wherein the aperture has a radius in the range of 2.5 mm to 3.5 mm, or
the ophthalmoscope comprises exactly eight lenses or exactly two planar discs, or
at least one lens is designed to be aspherical, or
at least one illuminated dot reticle is arranged in the at least one objective, centrally located on an optical axis of the at least one objective, or
the at least one illuminated dot reticle comprises at least one diffractive structure, wherein the at least one diffractive structure has an extent in the range from 25 μm to 75 μm, or
the at least one illuminated dot reticle is covered on the sensor side by a mask, or
the housing comprises at least one peripheral cylinder, wherein the at least one peripheral cylinder is designed blackened in the portion of the at least one illuminated dot reticle.

7. The ophthalmoscope according to claim 6, wherein the light of the eye can be transmitted in chronological order through an objective lens, the illuminated spot reticle, a meniscus lens, an object-sided air lens, an object-sided diverging lens, the aperture, a converging lens, an achromat, a sensor-sided meniscus lens, a sensor-sided diverging lens, and a protective glass, wherein the light of the eye can subsequently be impinged upon the at least one device for conversion, or the object lens or the object-sided meniscus lens or the achromat comprise flint glass, or the converging lens or the achromat comprise crown glass.

8. The ophthalmoscope according to claim 1, wherein at least one holding device for a head is provided , the holding device has a chin rest or a forehead support or is spaced from the at least one device for conversion in the range from 30 mm to 200 mm.

9. The ophthalmoscope according to claim 1, wherein the at least one device for conversion can be brought into data connection with at least one computer, wherein the at least one computer (34) can be brought into connection with a database.

10. The ophthalmoscope according to claim 1, wherein at least one illumination is arranged on the housing, wherein
the illumination of the eye via the at least one illumination is partially covered by the at least one objective, or
the at least one illumination is designed in the form of a white light illumination with a sunlight-like white tone with a characteristic color temperature between 5000 K and 6000 K or a color rendering index of at least 95% or a fluorescent illumination, or
the fluorescent illumination has a wavelength range between 450 nm and 510 nm or between 750 nm and 780 nm, or
at least one fluorescence filter is arranged between the at least one device for conversion and the eye, or
the ophthalmoscope comprises at least one status screen, wherein at least one electronic information can be visualized on the at least one status screen.

11. The ophthalmoscope according to claim 1 with a slit lamp.

12. A method for examining eyes, comprising the following steps:
rough adjustment of a patient in a holding device, in particular with an accuracy below 3 mm,
detection of a pupil and/or a vertex of a cornea of the eye via an image processing program, wherein it is particularly provided that the image processing program comprises an autofocus algorithm,
capturing at least one image of the eye by an ophthalmoscope according to claim 1, wherein the at least one image of the eye is captured on at least one device for conversion.

13. The method according to claim 12, wherein
movements of the eye below 3 mm are tracked by automated tracking, or an opening of an eyelid is detected via the image processing program, wherein when an opening is below 12 mm an indication is displayed on at least one status screen, or
the at least one image of the eye on the at least one device for conversion is read into or saved at at least one computer in a database on the at least one computer, or
the at least one image of the eye is evaluated or analyzed with regard to eye-specific characteristics, or
the at least one image of the eye is categorized at the at least one computer for standardization or norming, or
the eye comprises a contact lens and a tear fluid enriched with fluorescent dye, wherein the tear fluid is illuminated with fluorescent illumination, the tear fluid emits light in a wavelength range between 515 nm and 530 nm or between 825 nm and 835 nm, and a distribution of the emitted light of the tear fluid between the contact lens and the eye is recorded on the at least one device for conversion, or
the eye comprises at least one fluorescent dye and a blood vessel structure or a lymphatic vessel structure or a corneal epithelium of the eye is imaged on the at least one device for conversion or saved on the at least one computer, or
the fluorescent dye comprises fluorescein or indicyanine green, or
after a period of at least one day, the method is carried out again.

14. A computer program product comprising commands, which, when carried out by a computing unit, cause the computing unit to classify, for an ophthalmoscope for examining eyes, at least one image of an eye from a memory unit, which is or can be brought in data connection with the computing unit, wherein a classification is generated, based on a blood vessel structure or a corneal structure or a scar structure or an eye socket structure.

15. The computer program product according to claim 14, wherein an image overlay with automatic blood vessel detection or automatic limbus detection can be used to control the classification or a semi-automatic lesion detection and/or bidirectional measurements can be carried out.

16. The computer program product according to claim 14, wherein standardized images of at least one iris are saved in the memory unit.

17. The computer program product according to claim 14, wherein a color of a contact lens or an artificial iris is selected depending on a color of two iris, wherein the color of the contact lens or the artificial iris is matched to one of the two iris.

18. The computer program product according to claim 14, wherein the blood vessel structure is automatically detected or a quantification of changes in the blood vessel structure or the corneal structure is calculated via the image overlay.

19. The computer program product according to claim 14, wherein a red pixel density measurement is carried out, wherein the red pixel measurement integrates a total red portion of the at least one image, or calculates an area portion of the blood vessel structure at the at least one image.

20. The computer program product according to claim 14, wherein an automatic lesion detection of the eye is comprised, or an opening of the eye of at least 12 mm is detected and when the opening is less than 12 mm an electronic information is transmitted to the at least one status screen.
